(19) United States
(12) Patent Application Publication
(10) Pub. No.: US 2004/0101578 A1
(43) Pub. Date: May 27, 2004

(54) Compositon containg ginkgo biloba that inhibit angiogenesis and matrix metalloprotinase

(76) Inventors: Min-Young Kim, Daejeon-city (KR); Byung-Young Park, Daejeon-city (KR); Chang-Hee Moon, Daejeon-city (KR); Eun-Kyu Park, Daejeon-city (KR)

Correspondence Address: Joseph Hyosuk Kim, JHK Law, PO Box 1078, La Canada, CA, 91012-1078, US

(21) Appl. No.: 10/343,869

(22) PCT Filed: Aug. 3, 2001
(86) PCT No.: PCT/KR01/01325

(51) Int. Cl. A61K 35/78

(52) U.S. Cl. 424/752

(57) ABSTRACT

The present invention relates to a composition containing ginkgo biloba extract that inhibit angiogenesis and matrix metalloproteinases. The ginkgo biloba extract of the present invention suppress angiogenesis and activity of matrix metalloproteinases, so that it can be applied to medicine for disease related in angiogenesis and matrix metalloproteinases.

### BACKGROUND OF THE INVENTION

(a) Field of the Invention

The present invention relates to a composition comprising Ginkgo biloba leaf extract having anti-angiogenic and matrix metalloproteinase inhibitory activity, and more particularly, to a composition comprising Ginkgo biloba leaf extract that inhibits angiogenesis as confirmed by assay using human endothelial cells and by animal experiments, and that also inhibits matrix metalloproteinase activity.

(b) Description of the Related Art

The major components of the Ginkgo biloba leaf extract are flavonoids, biflavonoids, proanthocyanidins and terpene-lactones. The flavonoids comprise 0.5 to 1.8% of quercetin, isorhamnetin, 3O-methylmyristicin, and kaempferol. Among the biflavonoids, 0.4 to 1.9% of amentoflavone, bilobetin, 5-methoxybilobetin, ginkgetin and isoginkgetin are included. There are also 8 to 12% of proanthocyanidins and ginkgolide A, B, C as terpene-lactones from Ginkgo biloba leaves (Diamond et al., Arch Phys Med Rehabil, 5, 81668-78, 2000; DeFeudis FV, Pharmacological activities and clinical application. Paris, Elsevier, 1991).

Ginkgo biloba leaves are used for enhancement of blood circulation, and it is known that biflavonoids are responsible for the anti-inflammatory, anti-allergic, anti-rheumatic, and anti-cancer activity of Ginkgo biloba leaves (Korea patent KR09604025B1, KR09609183B1).

Angiogenesis is the process of generating new capillary blood vessels. Neovascularization is tightly regulated, and activation occurs in embryogenic development, tissue remodeling, wound healing and periodic cycles of corpus luteum development (Follkman and Cotran, Relation of vascular proliferation to tumor growth, Int Rev Exp Pathol 16 207-248, 1976).

The growth rate of endothelial cells is very low, and it takes several months to years to divide. However, there are some diseases caused by the failure of regulation of angiogenesis, abnormal growth of endothelial cells (Timar, J Pathol Oncol Res 6, 85-94, 2001). Pathological angiogenesis is related to angioma, angiofibroma, vascular deformity, and cardiovascular diseases such as atherosclerosis, synechia and edemic sclerosis; and opthalmological diseases such as neovascularization after cornea implantation, neovascular glaucoma, diabetic retinopathy, angiogenic corneal disease, macular degeneration, pterygium, retinal degeneration, retrolental fibroplasias, and granular conjunctivitis. Chronic inflammatory diseases such as arthritis; dermatological disease such as psoriasis, telangiectasis, pyogenic granuloma, seborrheic dermatitis and acne; and the proliferation and metastasis of cancer, are necessarily dependent on angiogenesis (D'Amato RJ and Adamis AP, Ophthalmol 102, 1261-1262, 1995; Arbiser JL, J Am Acad Derm 34, 486-497, 1996; O'Brien K. D. et al. Circulation 93, 672-682, 1996; Hanahan D and Folkman J, Cell 86, 353-364, 1996).

Since angiogenesis is closely related to initiation and progression of many diseases, many efforts have been made toward the development of anglogenesis inhibitors in order to prevent and/or treat those diseases.

In particular, angiogenesis in tumor cells plays very important roles in growth and metastasis. If angiogenesis and the migration of cancer cells are inhibited, they grow to a size of about 1-2 mm in diameter, and remain localized. (Folkman and Tyler, Cancer Invasion and metastasis, Biologic mechanisms and Therapy (S. B. Day ed.), Raven press, New York, 94-103,1977). However, when tumor cells approach adjacent capillary blood vessels, they are stimulated to secrete many angiogenic factors. Endothelial cells start to proliferate and migrate to the adjacent blood vessel, which induce a formation of the microvascular network.

Currently, a large variety of chemotherapies and immunotherapies are applied in the treatment of cancer, but the efficacy of the therapies is limited and nothing can successfully extend the life of cancer patients, due to the lack of anti-metastasis effects.

Angiogenesis is providing not only nutrients and oxygen, but also a way to entering the blood stream for metastasis (Polyerini P. J., Critical reviews in Oral Biology, 6, 230-247, 1995), and thus angiogenesis is essential for metastasis and growth of cancer. Therefore, metastasis could be blocked by inhibition of angiogenesis.

Many people are losing their eyesight all over the world because of various ocular diseases, which are also angiogenesis-dependent (Jeffrey MI and Takayuki A, J Clin Invest 103, 1231-1236, 1999).

For example, macular degeneration in elderly people, diabetic retinopathy, immature infant's retinopathy, neovascular glaucoma, and various corneal diseases are induced by angiogenesis (Adamis AP, Aiello LP and D'Amato RA, Angiogenesis 3, 9-14, 1999). Patients of diabetic retinopathy, a secondary disease of diabetes, become blind due to the infiltration of the capillary cells into the vitreous humor. Therefore, inhibition of angiogenesis is the basic therapeutic modality for these diseases.

Psoriasis is a chronic dermatological disease of erythema and scaling, caused by extremely active proliferation of skin cells: the normal dermis regenerates once a month while dermis of psoriasis patients regenerates more than once a week. Fast-growing cells requires sufficient blood supply, and angiogenesis is abnormally induced with psoriasis (Folkman J., J Invest Dermatol 59, 40-48, 1972). Therefore, inhibitors for angiogenesis can be applied to psoriasis as a new therapy.

One of the major events for inducing angiogenesis is a breakdown of the extracellular matrix before the formation of the capillary blood vessels. The most important enzyme of matrix degradation is matrix metalloproteinase (MMP), a family of over 20 proteins.

MMPs are endopeptidase, which degrade or proteolyze the components of the extracellular matrix such as collagen, proteoglycan, and gelatin, and are classified into four groups: collagenase, gelatinase, stromelysin, and membrane-type MMP.

Collagenase proteolyzes the triple helix interstitial collagen and gelatin, and it comprise interstitial collagenase (MMP-1), neutrophil collagenase (MMP-8) and collagenase-3 (MMP-13). These three enzymes share more than 50% sequence similarity, having two zinc-binding sites and one or two calcium binding sites in their core domain (Borkakoti et al., Nature Struct Biol, 1, 106-110, 1994; Bode, et al., EMBO J, 13, 1263-1269, 1994; Lovejoy et al., Science, 263, 375-377,1994).

Gelatinase can degrade denatured coilagen and type IV, V, VII and X collagen. There are two gelatinases, one is 72 kDa gelatinase-A (MMP-2) secreted from fibroblast, and the other is 92 kDa gelatinase B (MMP-9) secreted from mononuclear phagocytes. They specifically act on type IV collagen, the major component of the basement membrane (Murphy, G. et al., Biochem J, 258, 463-472, 1989; Stetler-Stevenson, W. G. et al., J Biol Chem, 264, 1353-1356, 1989). These enzymes are very important in cancer invasion and metastasis. As compared with MMP-2, MMP-9 comprises additional sequences with unknown functions between the C-terminal and catalytic domain (Wilhelm, S. M. et al., J Biol Chem, 17213-17221, 1989).

Stromelysins, originally known as proteoglicases, show a broad substrate spectrum, and stromelysin-1 (MMP-3), stromelysin-2 (MMP-10), stromelysin-3 (MMP-11), and matrilysin (MMP-7) are classified as stromelysin (Chin, J. R. et al., J Biol Chem, 260, 12367-12376, 1985; Whitham, S. E. et al., Biochem J, 240, 913-916, 1986).

Metaloelastinase (MMP-12), and membrane-type MMP such as MT1-MMP (MMP-14), MT2-MMP (MMP-15), and MT3-MMP (MMP-16), are also identified as enzymes in the MMP family.

Many enzymes in the MMP family have substrate specificity. The expression of MMP is induced under various physiological circumstances when remodeling of an extracellular matrix or other matrix composed of collagen is required.

Increased expression or activation of MMPs is observed in many pathological states, such as atherosclerosis, restenosis, MMP-dependentosteopathy, inflammation of the central nervous system, Alzheimer's disease, skin aging, rheumatoid arthritis, osteoarthritis, septic arthritis, corneal ulcer, synechia, bone disease, proteinuria, abdominal aortic aneurysm, regressive cartilage loss, myelinated nerve loss, liver fibrosis, nephrogromerula disease, germinal membrane ruptures, inflammatory bowel disease, gingivitis/periodontitis, senile macular degeneration, diabetic retinopathy, proliferate vitreous body retinopathy, immature retinopathy, eye inflammation, conical cornea, Sjogren syndrome, myopia, tumors in eyes, rejection of cornea implantation, angiogenesis, and cancer metastasis. (Woessner Jr., Annals NYAcad Sci, 732, 11-21, 1994; Warner et al., Am J Pathol, 158, 2139-44, 2001; Stetler-Stevenson, Surg Oncol Clin N Am, 10, 383-92, 2001).

In arthritis, a chronic inflammatory disease, cartilage is destroyed by proliferation of synovial and endothelial cells in the synovial cavity (Kocb AE, Polyerini PJ and Lcibovich SJ, Arth Rheum 29, 471-479, 1986; Stupack DG, Storgard CM and Cheresh DA, Braz J Med Biol Rcs 32, 578-581, 1999; Koch AE, Arthritis Rheum 41, 951-962, 1998). Stromelysins are known to be the major enzyme for disruption of cartilage, also playing an important role in activating procollagenases (Murphy, G. et al., Biochem J, 248, 265-268, 1987). Therefore, inhibition of MMP can prevent the progression of arthritis.

Collagenases, gelatinases and stromelysins are responsible for the degradation of the extracellular matrix. This is the major reason for visual power loss in many retinopathies (Bruns, F. R. et al., Invest Opthalmol and Visual Sci, 32, 1569-1575, 1989).

Gingivitis and periodontitis are induced by degradation of collagen in gingival tissue by collagenase secreted from the inflammatory cells in gingiva. Collagenases and stromelysins are identified in fibroblast from gingiva in inflammation, and the activity of the enzyme is dependent on the degree of inflammation (Beeley, N. R. A. et al., supra; Overall, C. M. et al., J Periodontal Res, 22, 81-88, 1987).

Recent reports have also shown that MMP-1 activity is highly induced in Alzheimer's disease, and MMP-1 and MMP-3 are involved in the pathophysiology of the disease (Leake A, Morris CM, & Whateley, J Neurosci Lett 291, 201-3, 2000; Yoshiyarrma Y, Asahina M, & Hattori T, Acta Neuropathol (berl), 99, 91-5, 2000).

By degradation of the basement membrane, MMPs are very important in many diseases, not only in cancer metastasis. However, no inhibitors have been developed for the treatment of these diseases. Since inhibitors for MMP and angiogenesis can be applied to many diseases, development of angiogenesis inhibitors for new therapies are expected.

Furthermore, these inhibitors should be administered to patients for a long period, and thus orally available compositions of minimal toxicity are needed to develop for new drugs.

### SUMMARY OF THE INVENTION

It is an object of the present invention to provide an orally available anti-angiogenic composition with reduced toxicity.

It is another object of the present invention to provide an anti-angiogenic composition comprising Ginkgo biloba leaf extract as the active ingredient.

It is a further object of the present invention to provide an MMP-inhibitory composition comprising Ginkgo biloba leaf extract as the active ingredient.

In order to achieve these objects, this invention provides a composition for inhibiting angiogenesis comprising Ginkgo biloba leaf extract.

Also, This invention provides a composition for inhibiting matrix metalloproteinase activity comprising Ginkgo biloba leaf extract.

### BRIEF DESCRIPTION OF THE DRAWINGS

FIG. 1 is a picture showing tube formation of human umbilical vein endothelial cells (HUVEC) as a control.

FIG. 2 is a picture showing tube formation of HUVEC treated with 0.4 μg/μl of Ginkgo biloba leaf extract.

FIG. 3 is a plot showing the anti-angiogenic effect of Ginkgo biloba leaf extract on the mouse Matrigel model.

FIG. 4 is a graph of the inhibition of angiogenesis by oral administration of Ginkgo biloba leaf extract in the mouse Matrigel model.

FIG. 5 is a picture showing chorioallantoic membrane assays confirming the anti-angiogenic effect of Ginkgo biloba leaf extract.

FIG. 6 is a picture showing tube formation of HUVEC treated with 1% DMSO.

FIG. 7 is a picture showing tube formation of HUVEC treated with 50 μM of quercetin.

FIG. 8 is a picture showing tube formation of HUVEC treated with 50 μM of kaempferol.

FIG. 9 is a picture showing tube formation of HUVEC treated with 50 μM of amentoflavon.

FIG. 10 is a picture showing tube formation of HUVEC treated with 1 mM of ginkgolide.

FIG. 11 is a graph showing inhibition of MMP-1 activity by Ginkgo biloba leaf extract.

FIG. 12 is a graph showing inhibition of MMP-2 activity by Ginkgo biloba leaf extract.

FIG. 13 is a graph showing inhibition of MMP-9 activity by Ginkgo biloba leaf extract.

FIG. 14 is a graph showing inhibitory effects of Ginkgo biloba leaf extract on cancer metastasis.

### DETAILED DESCRIPTION OF THE PRESENT INVENTION

Hereinafter, the present invention will be explained in detail.

The inventors recognize the inhibitory effect of Ginkgo biloba leaf extract on angiogenesis and matrix metalloproteinase.

Therefore, the present invention provides a Ginkgo biloba leaf extract that inhibits angiogenesis- and MMP-dependent diseases.

Ginkgo biloba leaf extract of the present invention can be purchased or prepared with conventional methods. The extraction can be performed by conventional methods of extraction from Ginkgo biloba leaves or dried powder of Ginkgo biloba. Commercially available Ginkgo biloba leaf extract and soft capsules contained Ginkgo biloba (no other additives are included) can also be used. The Ginkgo biloba leaf extracts include all kinds of Ginkgo biloba leaf goods containing more than 9.6 mg of ginkgoflavon glycosides in 40 mg of total weight, as described in guidelines from the Korea Food and Drug Administration.

One of the conventional extraction methods for Ginkgo biloba leaf extract is as follows.

10 to 20 L of an aqueous alcohol (for example, methanol, ethanol, butanol, etc.) or acetone is added to 1 kg of dried Ginkgo biloba leaves (green leaf or yellow lean or dried powder of Ginkgo biloba leaves. The mixture is allowed to extract at a temperature ranging from 60 to 80° C., for a period ranging from 30 min to 2 hours. The extraction process may be repeated 2 to 3 times with other solvents (chloroform, ethyl acetate, ketone, etc.). The resulting extract is concentrated to obtain a Ginkgo biloba leaf extract.

A detailed method for preparation of Ginkgo biloba leaf extract as undertaken for the present invention is as follows, but the preparation methods are not limited to this procedure.

Crude methanol extract was prepared from dried leaves of Ginkgo biloba, and then it was fractionated with a water/chloroform mixture. Ethylacetate was added to the aqueous phase, and dried with anhydrous Glauber's salt. The extracted material was dissolved in acetone and filtered, the filtrate was extracted with methylethylketone/acetone=1:1, then the organic phase was collected. It was concentrated, dissolved in ethanol, and filtrated to obtain Ginkgo biloba leaf extract. This Ginkgo biloba leaf extract preferably contains more than 15% of flavon-glycoside, terpene-lactone, and alkyl-phenol.

Ginkgo biloba leaf extract of the present invention inhibits tube formation of endothelial cells and angiogenesis in vivo Inhibition of tube formation was investigated by HUVEC tube formation assay, and antiangiogenic effect was confirmed by a CAM assay and a mouse Matrigel model. Furthermore, Ginkgo biloba leaf extract was shown to inhibit angiogenesis when it was administered orally.

The tube formation assay is an in vitro experimental method that is closely related to in vivo efficacy, and this method investigates the microvascular network of the human endothelial cell.

While the CAM assay is an in vivo assay using fertilized eggs, angiogenesis can be quantitatively measured by the mouse Matrigel assay.

The physiological activities of Ginkgo biloba leaf extract such as anti-inflammatory, anti-histamine, and anticancer activities are mediated by biflavonoids within the Ginkgo biloba, and more particularly, anticancer activity is measurable in cancer cells (Korean patent KR09604025B1, KR09609183B1). However, it has not been clear whether these compounds are responsible for angiogenesis. Endothelial cells, not cancerous cells, are responsible for angiogenesis, and the anticancer components of Ginkgo biloba are not necessarily the same as the anti-angiogenic compounds. When the inventor identified the compound for anti-angiogenic activity from Ginkgo biloba leaf extract, flavonoids were found responsible constituents for inhibition of angiogenesis.

Furthermore, the extract of Ginkgo biloba inhibits MMP, a family of essential enzymes for angiogenesis and cancer metastasis. The effect of Ginkgo biloba leaf extract on MMPs was investigated with MMP-1, MMP-2, and MMP-9, and it drastically inhibited activity of all three enzymes. The inhibitory effect of Ginkgo biloba leaf extract on MMPs is not, however, limited to these three enzymes.

It is therefore clear that Ginkgo biloba leaf extract of the present invention is available for a drug for angiogenesis- and/or MMP-dependent diseases since it inhibits angiogenesis and MMPs.

The angiogenesis-dependent diseases are cancer metastasis, angioma, angiofibroma, diabetic retinopathy, premature infant's retinopathy, neovascular glaucoma, angiogenic corneal disease, involutional macula, 1.2 macular degeneration, pterygium, retinal degeneration, retrolental fibroplasias, granular conjunctivitis, psoriasis, telangiectasis, pyogenic granuloma, seborrheic dermatitis, acne, and arthritis.

Overexpression of MMPs can induce cancer invasion and metastasis, atherosclerosis, restenosis, MMP-dependent-osteopathy, inflammation of the central nervous system, Alzheimer's disease, skin aging, corneal ulcer, synechia, bone disease, proteinuria, abdominal aortic aneurysm, regressive cartilage loss, myelinated nerve loss, liver fibrosis, nephrogromerula disease, ruptures in the germinal membrane, inflammatory bowel disease, gingivitis/periodontitis, senile macular degeneration, diabetic retinopathy, proliferative vitreous body retinopathy, immature retinopathy, inflammation in eyes, conical cornea, Sjogren syndrome, myopia, tumors in eyes, rejection of cornea implantation, rheumatoid arthritis, arthritis, and septic arthritis.

As mentioned above, Ginkgo biloba leaf extract of the present invention has inhibitory effects on angiogenesis and MMP activity. While MMPs are enzymes responsible for angiogenesis, anti-angiogenic activity of Ginkgo biloba leaf extract is not limited to MMP inhibition activity of the Ginkgo biloba. That is, MMPs are one of the factors for inducing angiogenesis, and Ginkgo biloba leaf extract can inhibit other factors for angiogenesis. Furthermore, the inhibitory of activity on MMP of Ginkgo biloba are not limited to inhibition of angiogenesis.

The present invention provides a composition containing Ginkgo biloba leaf extract for inhibiting angiogenesis. The above composition can be applied to inhibition of angiogenesis, cancer metastasis, and MMP enzyme activity.

The composition may be used by itself or Included with more than one pharmaceutical composition. A composition comprising Ginkgo biloba leaf extract can include more than one kind of pharmaceutical diluent, selected from the group consisting of saline, buffered saline, dextrose, water, glycerol, and ethanol, but the diluent is not limited. Appropriate diluents are listed in the written text of Remington's Pharmaceutical Science (Mack Publishing co, Easton Pa.).

Ginkgo biloba leaf extract composition may be applied differently according to the purpose of dosing and diseases. It should be understood that the amount of the active ingredient actually administered ought to be determined in light of various relevant factors including the condition to be treated, the severity of the patient's symptoms, co-administration with other drugs (e.g., chemotherapeutic agents), age, sex, body weight of the individual patient, food, dosing time, the chosen route of administration, and the ratio of the composition.

More preferably, a daily dose of a Ginkgo biloba leaf extract is preferable from about 5 to 800 mg, and most preferably 40 to 400 mg. In general, 0.1 to 20 mg/kg, preferably 0.2 to 10 mg/kg of Ginkgo biloba leaf extract can be administrated in a single or in 1-3 divided doses per day, even though the exact dose and route of administration are adjusted to the type and severity of disease.

The composition comprising Ginkgo biloba leaf extract of the present invention can be administered via oral or parenteral routes. Parenteral dosing means the administration of a drug through a route other than oral, which includes rectal, intravenous, intraperitoneal and intramuscular, intra-arterial, transdermal, nasal, inhalation, ocular, and subcutaneous introduction.

Pharmaceutical formulations containing Ginkgo biloba leaf extract may be prepared in any form, such as oral dosage form, injectable solution, or topical preparation. The formulation can be preferably prepared for oral and injectable administration (true solution, suspension, or emulsion) and most preferably in oral form such as tablet, capsule, soft capsule, aqueous medicine, pill, granule, and the like.

In preparing the formulation, the active ingredients of Ginkgo biloba leaf extract are filled in the soft capsule without any excipient, or formed as an appropriate formulation after mixing or diluting with a carrier. Examples of suitable carriers are starches, water, saline, Ringer's solution, dextrose, and any ingredients described in previous reports (e.g. Remington's Pharmaceutical Science, Mack Publishing Co., Easton Pa.).

The following examples are intended to further illustrate the present invention. However, these examples are shown only for better understanding the present invention without limiting its scope.

### EXAMPLE 1

Ginkgo biloba leaf extract was purchased from Hwail Pharmaceutical Co. and used in the following examples: it contains more than 9.6 mg of ginkgoflavon in the 40 mg of total weight as described in the guidelines from the Korea Food and Drug Administration.

<TEST 1> Effects of Ginkgo biloba Leaf Extract on Tube Formation of HUVEC

The effect of Ginkgo biloba leaf extract on angiogenesis was investigated. The effect on the formation of the microvascular network was observed in vitro with human endothelial cells.

In order to do the tube formation assay, blood vessel endothelial cells, that Is, human umbilical vein endothellal cells (HUVECs), were isolated. HUVECs were isolated from freshly obtained cords after cesarean section, and they were grown and identified by immunocytochemial staining with anti-Factor Vil antibody. HUVECs grown with Matrigel (BD Bioscience, Bedford, Mass., USA), were treated with 0.4 μg/μl of the above Ginkgo biloba leaf extract (Hwa II Pharmaceutical Co., LTD, Korea) of the EXAMPLE 1, and further incubated at 37° C. for 1618 hrs. As a control, the procedure was repeated without Ginkgo biloba leaf extract.

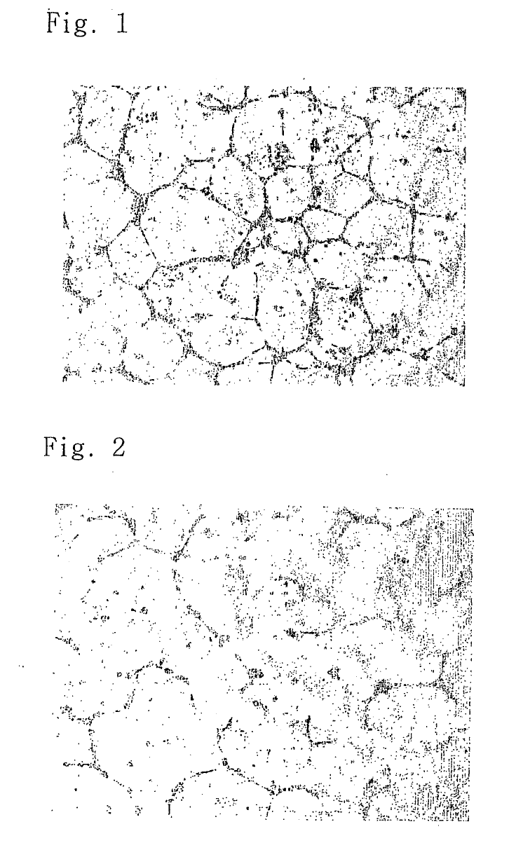

FIG. 1 shows that a tubular network is formed as a process of neovascularization, when they are grown with Matrigel.

FIG. 2 is the HUVECs grown in Matrigel treated with 0.4 μg/μl of Ginkgo biloba leaf extract, which shows that the microvascular network was disconnected. The area of the tube was determined by the image analysis program Image-Pro Plus (Media Cybernetics, USA), and as summarized in Table I, tube formation after treatment of Ginkgo biloba leaf extract was inhibited by about 60% as compared with the untreated control.

TABLE 1

| | Tube area | % |
|---|---|---|
| Control | 12.75 | 100 |
| 0.4 μg/μl Ginkgo biloba leaf extract | 5.05 | 39.6 |

<TEST 2> Animal Experiment for Angiogenesis (Mouse Matrigel Model)

The anti-angiogenic activity of Ginkgo biloba leaf extract was quantitatively measured in a mouse Matrigel model. A 0.4 ml portion of Matrigel mixed with 50 ng/ml of basic fibroblast growth factor (bFGF) and 50 units of heparin was implanted by subcutaneous injection in 6 to 8-week-old C57BL/6 female mice. After 3-5 days, Matrigel was recovered from excised skin of each mouse, the amount of hemoglobin (Hb) in the Matrigel was measured as a control with a Drabkin kit (Sigma Chemical Co., St. Louise, Ml, USA, Cat. No. 525), a reagent for determination of total hemoglobin in blood.

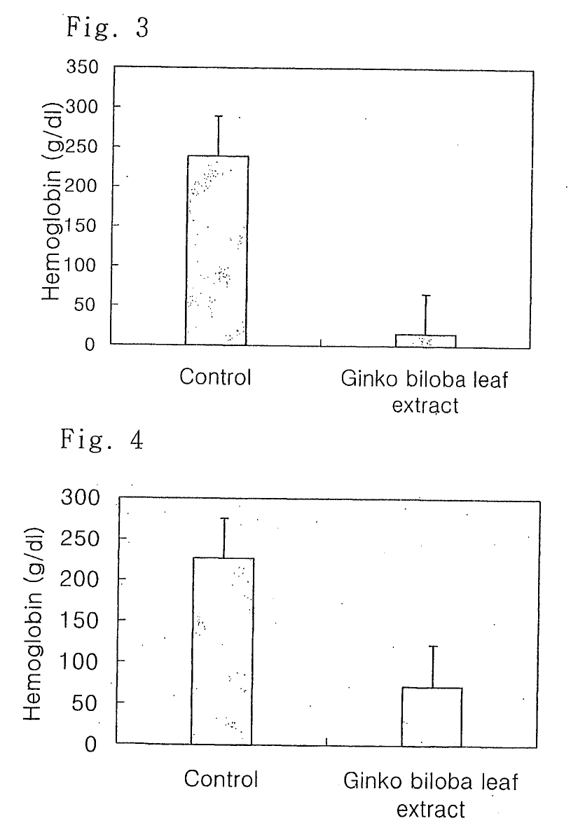

The same experiment was done with Matrigel mixture of the EXAMPLE 1 comprising Ginkgo biloba leaf extract (38 μg), and hemoglobin content of the treated group was compared with that of the control group. As shown in FIG. 3 and Table 2, the hemoglobin content of the treated mice was remarkably reduced as compared with that of the control mice, which means that angiogenesis was inhibited by about 94%.

TABLE 2

| | Hemoglobin (g/dL) |
|---|---|
| Control | 239 ± 329 |
| Ginkgo biloba leaf extract | 15.7 ± 8.1 |

In order to test the activity of orally administrated Ginkgo biloba leaf extract, the following experiment was undertaken.

Ginkgo biloba leaf extract (0.4 mg/mouse) was orally administered to C57BL/6 mice, and 0.4 ml of Matrigel containing 50 ng/ml of basic fibroblast growth factor (bFGF) and 50 units/ml of heparin was implanted by subcutaneous injection at 14 hrs after treatment. Ginkgo biloba leaf extract (0.4 mg/mouse) was orally administered twice per day for 3 days. After 3 days, the Matrigel was removed and the amount of hemoglobin in the Matrigel was determined.

As shown in FIG. 4 and Table 3, the Ginkgo biloba leaf extract-treated group showed a lower level of hemoglobin in the Matrigel, at about 68% of that of the control group. That is, the Ginkgo biloba leaf extract also showed anti-angiogenic activity when it was administered orally.

TABLE 3

| | Hemoglobin (g/dl) |
|---|---|
| Control | 226 ± 229 |
| Ginkgo biloba leaf extract | 72 ± 55 |

<TEST 3> Angiogenesis Assay with Chorioallantoic Membrane Assays (CAM Assay)

Fertilized chicken eggs were incubated for three days in a humidified incubator of over 70% humidity. From each egg, 2-3 ml of albumin was aspirated with a syringe with a 26-gauge needle, and the egg was sealed with transparent adhesive tape. A window of 1×1 cm was made in the middle of the embryo with a drill. An aliquot of 60 μof Ginkgo biloba leaf extract was applied to sterile Thermanox discs (Miles Scientific) and allowed to air dried, and the discs were applied to the chorioallantoic membrane surface through the window and covered with tape. The embryos were incubated for three days at 37° C. in a humidified incubator. As a control, 15 μl of physiological saline solution was loaded to a disc instead of Ginkgo biloba leaf extract, the same procedure was repeated and the resulting blood vessels were observed and compared with treated eggs. An appropriate volume of a lipid emulsion was injected into the embryo chorioallantois using a 26-gauge needle so that the vascular network of the chorioallantoic membrane stood out against the white lipid background.

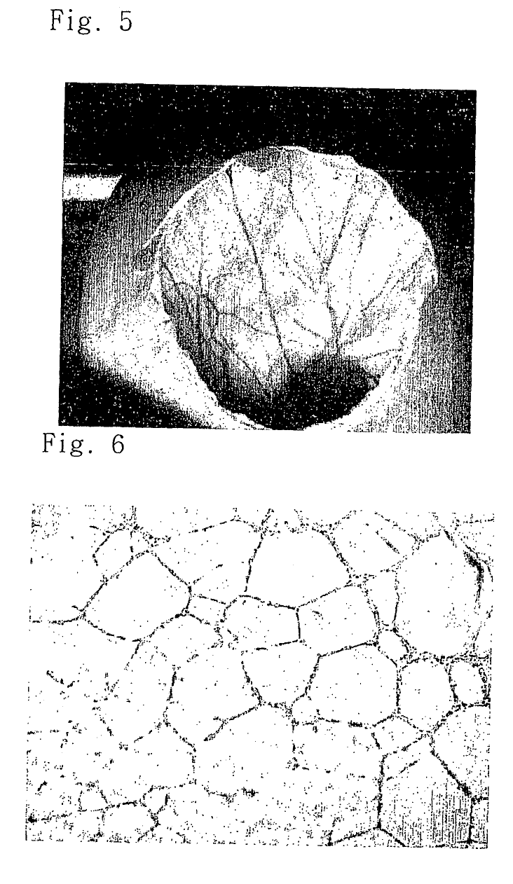

In the control group (n=20), capillary vessel formation was not affected in 90% of the embryo, while the inhibition of vessel formation in the disc (brighter part of the picture) with Ginkgo biloba was significant as shown in FIG. 5, and the inhibition of the blood vessel formation of the chorioallantois was observed in all the treated eggs (n=20, 100%).

<TEST 4> Identification of Active Component of Ginkgo biloba Leaf Extract for Anti-Angiogenic Activity

Among flavonoids, biflavonoids and terpene-lactones from Ginkgo biloba leaf extract, compounds having anti-angiogenic activity were identified by the following experiments.

Representative compounds from Ginkgo biloba, quercetin (Sigma Cat No. Q0125) and kaempferol (Indofine Chemical Cat No. K-102) as flavonoids, amentoflavon (Indofine Chemical Cat No. 021101S) as a biflavonoid and ginkolide A (Sigma Cat No. G4028) as a terpene-lactone, were subjected to the tube formation assay as described in EXAMPLE 1.

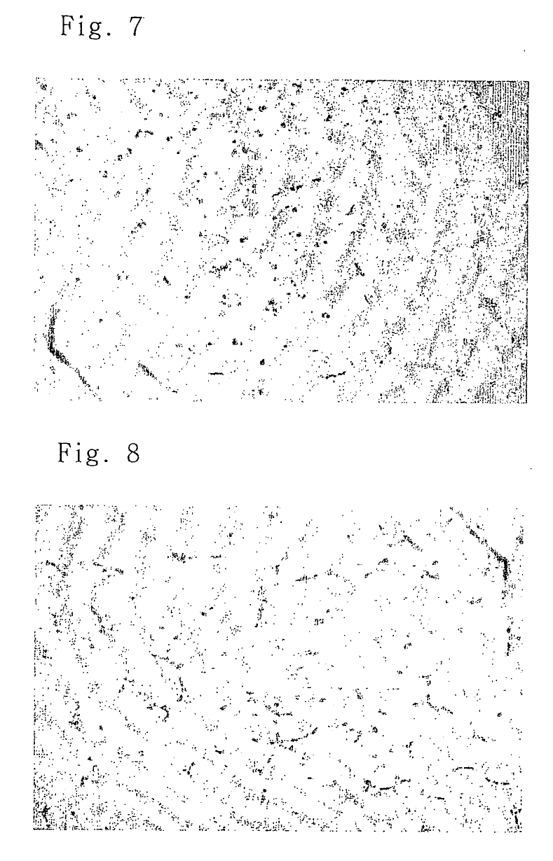

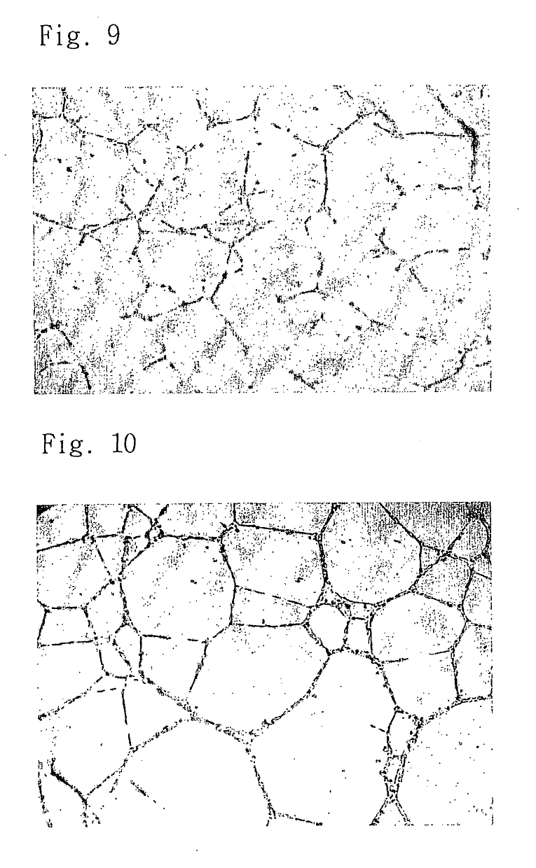

Since the above chemicals are not soluble in water, they were dissolved on dimethyl sulfoxide (DMSO), and in order to exclude the effect of solvent for these compounds, HUVECs treated with the same amount of DMSO were used as a control. FIG. 6 is a picture of the 1% DMSO-treated HUVEC control, and FIGS. 7-10 are pictures showing the effect on tube formation of individual compounds (quercetin, kaempferol, amentoflavon, and ginlkolide A, respectively). At 50 μM concentration, the order of inhibitory effect on HUVEC tube formation was quercetin>kaempferol>amentoflavon>ginkgolide A. Ginkgolide A was not effective even at 1 mM concentration.

The area of the tube was analyzed by Image-Pro Plus®, and the results are summarized in Table 4. The percent inhibition of the tube formation by quercetin, kaempferol, and amentoflavon was 93, 73, and 45, respectively. Therefore, not biflavonoids but flavonoids such as quercetin and kaempferol, are the major component for angiogenesis inhibition in Ginkgo biloba leaf extract.

TABLE 4

| Treatment | Area of the tube | % |
|---|---|---|
| DMSO control | 88 | 100 |
| Quercetin | 6.6 | 7.5 |
| Kaempferol | 24 | 27 |
| Amentoflavon | 48 | 55 |

<TEST 5>

(1) Preparation of MMP

MMP-1, MMP-2, and MMP-9 were cloned and prepared from insect cells (Sf21 insect cell) by using a Baculovirus system.

MMP-2 cDNA (GENEBANK No. XM_{—}048244) was cloned to a pBlueBac4.5 transfer vector (Invitrogen, Cat no. V1995-20), and then transfected to Sf9 cells with a Bac-N-Blue Transfection Kit (Invitrogen, Cat no. K855-01). Sf21 cells were incubated with a TNM-FH (Sigma, St. Louis, Mo., U.S.A) media containing 10% fetal bovine serum at 27° C., then harvested and re-suspended at a concentration of 10⁷ cell/ml. The cell suspension was incubated with a virus containing the cloned gene for 1 hr at room temperature. Infected Sf21 cells were grown for 72 hrs and the medium was recovered, and the MMP-2 was purified with a gelatin-sepharose affinity column from the recovered medium MMP-1 (GENEBANK NO. XM_{—}040735) and MMP-9 (GENEBANK NO. XM_{—}009491) were prepared from corresponding genes as previously described, MMP-1 was purified with SP-sepharose, and MMP-9 was purified by gelatin-sepharose affinity chromatography.

(2) Inhibition of MMP Activity

In order to investigate MMP inhibition by Ginkgo biloba leaf extract, MMP enzyme activity was assayed by a spectrofluorometric method (Perkin-Elmer LS50B).

Purified MMP-1, MMP-2, and MMP-9 were used after activation with 1 mM APMA before assay.

The substrate for MMP-1 and MMP-9 was 2,4-dinitrophenyl-Pro-Leu-Ala-Leu-Trp-Ala-Arg (SEQ ID No. 1), and Mca-Pro-Leu20-13-Gly-Leu-Dap(Dnp)-Ala-Arg-NH₂ (SEQ ID No. 2:BACHEM, Cat. No. M-1895) was used as a substrate for MMP-2.

As a control, 10 nM MMP-1 and 10 μM substrate (Sequence No. 1) were mixed in 2 ml of reaction buffer (50 mM Tricine (pH 7.5), 10 mM CaCl₂, 200 mM NaCl) in a 2 ml cuvette. Fluorescence intensity was measured every 2 min for 20 min at room temperature with a spectrofluorometer under an excitation wavelength of 280 nm and an emission wavelength of 360 nm.

Ginkgo biloba leaf extract (25 μg/ml) suspended in water and 10 nM MMP-1 was added to a reaction buffer containing a substrate, and fluorescence intensity was measured in the same manner.

Activity for MMP-2 or MMP-9 was also assayed, and fluorescence intensity was measured as previously mentioned.

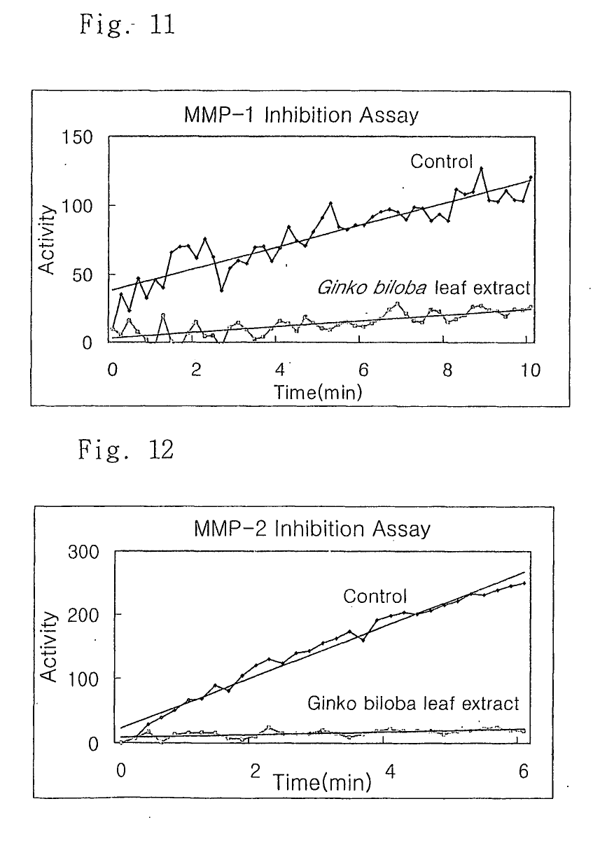

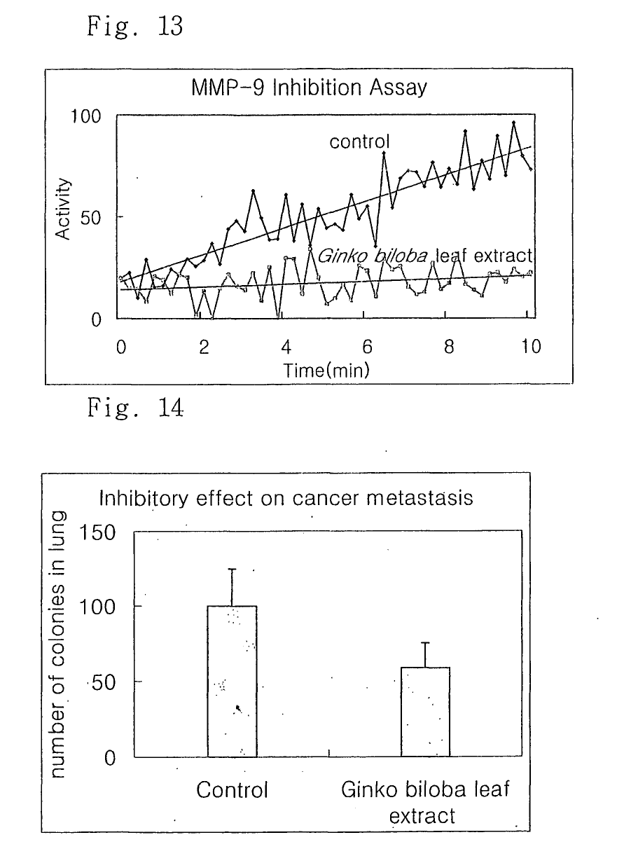

FIGS. 11, 12 and 13 are diagrams of activity of MMP-1, MMP-2, and MMP-9. As shown in FIG. 11, 76% of MMP-1 activity was inhibited by Ginkgo biloba leaf extract. The inhibition of MMP-2 and MMP-9 by Ginkgo biloba leaf extract was 93% (FIG. 12) and 90% (FIG. 13), respectively.

<TEST 6> Inhibition of Cancer Metastasis by Ginkgo biloba Leaf Extract

C57BL/6 male mice in age of 6 to 7 weeks were divided into two groups of 6 mice per group, and 5×10⁴ B16BL6 cells were injected into each mouse through the tail vein. After that, 0.2 ml of water was given to mice in the control group, and Ginkgo biloba leaf extract, 0.5 mg/0.2 ml/mouse, was orally administrated twice per day for 3 weeks to mice in the experimental group. Three weeks after injection, the mice were sacrificed and the number of colonies on the surface of lungs was counted under microscope. When the mice were daily treated with 1 mg of Ginkgo biloba per mouse, number of B16BL6 melanoma colonies in lungs from treated mice were 41% to those from control mice (Table 5).

TABLE 5

| Preparation | Colonies in lung | Inhibition (%) |
|---|---|---|
| Control | 100 ± 25 | 0 |
| Ginkgo biloba leaf extract | 59 ± 16 | 41 |

As previously mentioned, Ginkgo biloba leaf extract of the present invention inhibits angiogenesis and matrix metalloproteinase activity. Flavonoids such as quercetin and laempferol are identified as active constituents of Ginkgo biloba for inhibiting angiogenesis and cancer metastasis inhibition. Based on that, Ginkgo biloba leaf extract can be used as a new drug for treatment of angiogenesis- and/or MMP-dependent diseases.

What is claimed is:

1. A composition for inhibiting angiogenesis comprising a Ginkgo biloba leaf extract.

2. The composition according to claim 1, wherein the composition is used for treatment of a angiogenesis-associated disease selected from the group consisting of cancer metastasis, angioma, angiofibroma, diabetic retinopathy, premature infant's retinopathy, neovascular glaucoma, corneal disease induced by angiogenesis, involutional macula, macular degeneration, pterygium, retinal degeneration, retrolental fibroplasias, granular conjunctivitis, psoriasis, telangiectasis, pyogenic granuloma, seborrheic dermatitis, acne, and arthritis.

3. The composition according to claim 1, wherein the leaf extract comprises flavonoid compounds.

4. A composition for inhibiting matrix metalloproteinases(MMP) activity comprising a Ginkgo biloba leaf extract.

5. The composition according to claim 4, wherein the MMP is at least one selected from the group consisting of matrix metalloproteinase-1, matrix metalloproteinase-2 and matrix metalloproteinase-9.

6. The composition according to claim 4, wherein the composition is used for treatment of a MMP-associated disease selected from the group consisting of cancer metastasis, atherosclerosis, restenosis, MMP-dependent osteopathy, inflammation of the central nervous system, Alzheimer's disease, skin aging, corneal ulcer, synechia, bone disease, proteinuria, abdominal aortic aneurysm, regressive cartilage loss, myelinated nerve loss, liver fibrosis, nephrogromerula disease, germinal membrane rupture, inflammatory bowel disease, gingivitis/periodontitis, senile macular degeneration, diabetic retinopathy, proliferate vitreous body retinopathy, immature retinopathy, eye inflammation, conical cornea, Sjogren syndrome, myopia, eye tumor, rejection in cornea implantation, rheumatoid arthritis, arthritis, and septic arthritis.

7. The composition according to claim 4, wherein the composition is formulated in an oral dosage form, injectable solution, or topical preparation.


SEQUENCE LISTING (2 sequences)

SEQ ID NO: 1 - protein, 7 aa, Artificial
  Substrate for MMP-1 and MMP-9
PLALWAR

SEQ ID NO: 2 - protein, 6 aa, Artificial
  Substrate for MMP-2
PLGLAR